(19) United States
(12) Patent Application Publication
(10) Pub. No.: US 2020/0262937 A1
(43) Pub. Date: Aug. 20, 2020

(54) HIGH MOLECULAR WEIGHT CHITOSAN, PROCESS FOR OBTAINING AND USES THEREOF

(71) Applicant: ASSOCIATION FOR THE ADVANCEMENT OF TISSUE ENGINEERING CELL BASED TECHNOLOGIES & THERAPIES (A4TEC)-, Braga (PT)

(72) Inventors: Rita LÓPEZ CEBRAL, Guimarães (PT); Tiago José QUINTEROS LOPES HENRIQUEZ DA SILVA, Ponte de Lima (PT); Joaquim Miguel ANTUNES CORREIA DE OLIVEIRA, Braga (PT); Ramón NOVOA CARBALLAL, Braga (PT); Rui Luís GONÇALVES DOS REIS, Porto (PT)

(21) Appl. No.: 16/652,001

(22) PCT Filed: Sep. 27, 2018
(86) PCT No.: PCT/IB2018/057514

§ 371 Date: Mar. 27, 2020

(30) Foreign Application Priority Data
Sep. 27, 2017 (PT) 110309

(51) Int. Cl. C08B 37/08; A61K 47/36

(52) U.S. Cl. CPC C08B 37/003; A61K 47/36

(57) ABSTRACT

The present disclosure relates to a method for obtaining a high molecular weight chitosan with a lower acetylation degree and its use in human or veterinarian medicine. More specifically, to the obtaining of this biomaterial by means of a simpler process, with reduced energy costs, when compared with conventional procedures.

### TECHNICAL FIELD

The present disclosure relates to a method for obtaining a high molecular weight chitosan with a lower acetylation degree and its use in human or veterinarian medicine. More specifically, for obtaining this biomaterial by means of a simpler process, with reduced energy costs, when compared with conventional procedures.

### BACKGROUND ART

The exploitation of polysaccharides like chitin and chitosan from marine biomass has attracted great interest as an eco-friendly and sustainable strategy.

Chitin (Ch) is the second most abundant natural polymer, after cellulose. Structurally, Ch is composed by N-acetyl-D-glucosamine and D-glucosamine monomers, bonded by β-D-(1 [symbol] 4) linkages. Chitin has appealing properties from a biomedical point of view, such as biocompatibility, tumour cell growth suppression, acceleration of wound healing and antimicrobial activity. Moreover, chitin is highly biodegradable, due to the presence of hydrolytic enzymes in the human body (namely lysozyme) able to break the glycosidic bonds present in the chitin chain. Nevertheless, the limited solubility of Ch in almost all common solvents is an important drawback for industrial exploitation.

Depending on the source, Ch will present α- and β-form. The first is the most abundant type of chitin in nature, found in the exoskeleton of different crustaceans; while β-chitin constitutes the endoskeletons of diverse molluscs. In both types of chitins the chains are organized in sheets and held together by intra-sheet hydrogen bonds. However, no inter-sheet hydrogen bonds are present in the β-Ch structure. This may be the reason of its higher affinity towards solvents, its swelling in water or alcohol without affectation of the crystallinity or its larger reactivity. Although, as previously stated both α- and β-Ch are insoluble in most common solvents.

Chitosans (Cht) is the de-acetylated derivative of chitin. Diverse characteristics make of chitosans an interesting biomedical material, such as biodegradability, biocompatibility, mucoadhesivity, antimicrobial and anti-oxidant activity, lack of toxicity, haemostatic action and cationic nature. The α or β character of the original Ch will condition the characteristics of the obtained Cht.

β-Ch is the structural polysaccharide in squid pens. This raw material comes in tons of weight of residues per year from the fishing industry. This means tons of useless squid pens that should somehow be addressed/eliminated in order to avoid environmental problems.

Molecular weight is a very important parameter in biomaterials science. As it will significantly condition the characteristic of the biomedical formulations obtained using the biomaterial. The extreme conditions of the conventional extracting procedures (reaction with concentrated NaOH at high temperatures during long reaction times or repetitive cycles) provoke the rupture of the polysaccharide chains, ultimately reducing the molecular weight of chitosan. As a consequence, normally low (Mw 50-199 KDa) or medium (Mw 200-349 KDa) molecular weight chitosans are obtained.

The previously mentioned methodologies also imply a high energetic cost.

These facts are disclosed in order to illustrate the technical problem addressed by the present disclosure.

### GENERAL DESCRIPTION

The present disclosure relates to a method for obtaining a high molecular weight chitosan with a lower acetylation degree and its use in human or veterinarian medicine. More specifically, for obtaining this biomaterial, namely membranes, by means of a simpler process, with reduced energy costs, when compared with conventional procedures (see FIG. 6).

β-Cht presents a series of structural advantages when compared with α-Cht. β-Cht is obtained from β-Ch de-acetylation. Ch is considered to be transformed into Cht when de Degree of Acetylation (DA) is below 40%. In order to be cost-effective and competitive, Cht should be obtained in a reproducible manner, following a procedure as simple, fast, eco-friendly and low-cost as possible.

β-Ch is present in nature, mainly, as structural component forming the endoskeletons of molluscs. More specifically, squid pens endoskeletons are formed by β-Ch. Together with Ch other natural molecules form part of squid pens. More specifically, it has been described that as an average squid pens are composed by Ch (38%), proteins (61%) and some minerals (1%).

It should be taken into account the important fact that squid pens constitute a potential environmental problem. Due to the tons of useless squid pens resulting from fishing industry activity.

An aspect of the present disclosure relates to a method for obtaining a high molecular weight chitosan comprising the following steps:
providing milled squid pen with a particle size between 63 to 125 μm; preferably milling squid pen and selecting the milled squid pen; with a particle size between 63-125 μm;
reacting NaOH with the particles selected in the previous step, for at least 1.5 hrs at 75° C., under stirring and in a N₂ atmosphere;
obtaining a high molecular weight chitosan.

In an embodiment, the selection of the milled squid pen can be performed with a sieve, or a plurality of sieves to obtain the desired size.

One of the problems in the industrial production of chitosan is the high-water consumption used in washing and neutralization thereof. The method described in the present subject-matter reduces substantially water consumption, this advantage leading to both a lower water consumption costs (in up-stream), and lower subsequent effluent treatment costs (in down-stream).

In an embodiment for better results, the amount of selected particles is between 4-20 g, preferably 5-10 g.

In an embodiment for better result, the amount of NaOH is 200 mL.

In an embodiment for better results, the NaOH is a solution of 50% (v/v) NaOH.

In an embodiment for better results, the reaction time is between 1.5-3.5 hrs, preferably 2 hrs.

In an embodiment for better results, the chitosan of the present disclosure may be obtained by means of a one-step procedure, prolonged during a short period of time 2 hours.

In an embodiment for better results, the obtained chitosan is frozen at −80° C. and/or freeze-dried for 3 days.

In an embodiment for better results, the method may further comprise a previous washing of the squid pen to eliminate gross impurities.

In an embodiment for better results, the method may further comprise the cleaning of the obtained chitosan.

Another aspect of the present disclosure relates to a chitosan comprising a molecular weight of at least 500-1200 kDa and an acetylation degree between 5-40%, preferably comprising an acetylation degree between 5-30%.

In an embodiment for better results, the chitosan may comprise an acetylation degree up to 25%; preferably an acetylation degree up to 20%, more preferably an acetylation degree up to 15%; even more preferably an acetylation degree up to 10%, even more preferably an acetylation degree up to 5%.

In an embodiment for better results, the chitosan may comprise a molecular weight of at least 600 kDa, preferably 700 kDa, more preferably 900 kDa.

In an embodiment for better results, the chitosan may comprise a molecular weight of at least 1000 kDa, preferably 1200 kDa, more preferably 1500 kDa.

In an embodiment for better results, the chitosan may comprise a molecular weight between 350-1500 kDa, preferably between 500-1200 kDa, more preferably between 800-1100 kDa.

In an embodiment for better results, the chitosan may comprise protein concentration up to 0.1 mg/ml; preferably 0.08-0.025 mg/ml.

In an embodiment for better results, the chitosan may be β-chitosan.

Another aspect of the present disclosure relates to the use of the high molecular weight chitosan of the present disclosure in medicine or veterinary, namely for use in human or veterinarian regenerative medicine and/or tissue engineering.

In an embodiment for better results, the chitosan of the present disclosure may be used in the treatment or prevention of bone, cartilage, osteochondral, joint, muscle, musculoskeletal, ligament, tendon, connective, ocular, skin, vascular, lymphatic, liver, kidney, spleen, pancreas, reproductive organs, peripheral nerve, spinal cord or brain diseases.

In an embodiment for better results, the chitosan of the present disclosure may be used in the treatment or prevention of human or veterinarian wound healing.

In an embodiment for better results, the chitosan of the present disclosure may be use as a drug delivery system or as a viscosupplement.

Another aspect of the present disclosure relates to a pharmaceutical composition comprising the high molecular weight chitosan of the present disclosure combined with (an) active substance(s).

Another aspect of the present disclosure relates to a pharmaceutical composition comprising the high molecular weight chitosan of the present disclosure in a therapeutically effective amount or as a pharmaceutically acceptable excipient.

In an embodiment for better results, the composition may comprise 0.1-50% of said chitosan, preferably 0.5-30% (w/v) of chitosan; preferably 0.5-10% (w/v) of chitosan.

In an embodiment for better results, the composition may further comprise a second polysaccharide(s), in particular (a) seaweed polysaccharide(s), (a) beta glucan(s), (a) galactomannan(s), (a) mucilage(s), (a) cellulose(s), (a) inulin(s), (a) pullulan(s), (a) dextrin(s), (a) starch(es), (a) glycosaminoglycan(s), or mixtures thereof.

In an embodiment for better results, the composition may further comprise (a) protein(s), (a) growth factor(s), (a) digestive enzyme(s), (a) metabolic enzyme(s) hormone, a drug, or mixtures thereof, in particular albumin.

In an embodiment for better results, the composition may further comprise cell culture media or buffered media, in particular a liquid, semi-solid, solid or gas cell culture media or a natural, synthetic or semi-synthetic cell culture media.

In an embodiment for better results, the composition may be administrated by topical, enteral or parenteral administration.

In an embodiment for better results, the composition may further comprise a hydrogel or a plurality of hydrogels.

In an embodiment for better results, the composition may take the form of a scaffold, a bead, a microsystem or a nanosystem.

Another aspect of the present disclosure relates to a hydrogel comprising at least 0.1% of the high molecular weight chitosan of the present disclosure.

Another aspect of the present disclosure relates to a membrane comprising the high molecular weight chitosan of the present disclosure and/or the composition of the present disclosure.

In an embodiment, oxygen reacts with carbohydrates. The oxidation of glucose molecules provokes the breakdown of carbohydrates, generating energy. In order to avoid this breakdown, and contribute to the obtaining of high molecular weight chitosan, the reaction was conducted under N₂ atmosphere, which is an inert atmosphere.

In an embodiment, the efficiency of the reaction was 26.22% ±0.74 (w/w). Normally the average content of chitin in squid pens was described as 38%. This would mean a real efficiency of around 70% (w/w).

The chemical features of chitosan clarified by Fourier Transform Infrared spectroscopy (FTIR). Nuclear Magnetic Resonance (NMR) confirmed that the reaction product was chitosan, also demonstrating its purity. NMR also indicated an average DA of 22.77% (±0.68).

In an embodiment, size exclusion Chromatography-Right angle Laser-Light Scattering (SEC-MALLS) was used to determine the weight average molecular weight (Mw) and the number average molecular weight (Mn) of the obtained chitosans. The results were 1,003.94 (±81.20) kDa and 575.91 (±9.77) kDa, respectively.

In an embodiment, high molecular weight chitosan is obtained from natural organisms. Preferably, from marine origin organism. More preferably, from squid pens.

In a preferred embodiment, the obtained chitosan is β-chitosan.

In an embodiment, the average DA is defined as 22.77% ±0.68.

In an embodiment, the average Mw is defined as 1,003 ±81 kDa.

In an embodiment, the average Mn is defined as 575.9 ±9.8 kDa.

In an embodiment, the performance of an additional reaction cycle led to chitosan with lower DA (5.66%±0.15), with a reaction efficiency of 80.3%±1.6.

In an embodiment, the invention may comprise chitosan with DA defined as 5-39%, preferably 5-30%, more preferably 15-25%.

In an embodiment, the invention may comprise chitosan with a Mw between 350-1500 KDa, preferably 500-1200 kDa, more preferably 800-1100 kDa.

In an embodiment, high molecular weight chitosan or its derivatives may be used in the treatment or prevention of bone, cartilage, osteochondral, joint, muscle, musculoskeletal, ligament, tendon, connective, ocular, skin, vascular, lymphatic, liver, kidney, spleen, pancreas, reproductive organs, peripheral nerve, spinal cord or brain diseases, human or veterinarian.

In an embodiment, high molecular weight chitosan or its derivatives may be used in the preparation of hydrogels or other scaffolds for tissue engineering and regenerative medicine, human or veterinarian.

In an embodiment, high molecular weight chitosan or its derivatives may be used in the preparation of hydrogels, nanoparticles or other vehicles for human or veterinarian drug/cell delivery.

In an embodiment, high molecular weight chitosan or its derivatives may be used in the preparation of hydrogels, nanoparticles or other vehicles for human or veterinarian diagnosis.

In an embodiment, high molecular weight chitosan or its derivatives may be used as pharmaceutical excipients, preferably fillers, binders, disintegrants, coatings, sorbents, anti-adherents, lubricants, glidants, preservatives, antioxidants, flavouring agents, sweeting agents, colouring agents, solvents and co-solvents, buffering agents, chelating agents, viscosity imparting agents, surface active agents or humectants.

In an embodiment, high molecular weight chitosan or its derivatives may be used as human or veterinarian viscosupplements.

In an embodiment, high molecular weight chitosan or its derivatives may be used in the prevention or treatment of tissue diseases or defects, or wound healing, both human and veterinarian.

In an embodiment, the composition may comprise 0.1-50% (w/v) of chitosan or its derivatives; preferably 0.5-30% (w/v) of chitosan or its derivatives; preferably 0.5-10% (w/v) of chitosan or its derivatives.

In an embodiment, the composition may further comprise other polysaccharides. Preferably the polysaccharide may be selected from the following list: glycosaminoglycans, cellulose, alginate, fucoidan, dextrin, carrageenan, gellan gum, guar gum or mixtures thereof.

In an embodiment, the composition may further comprise proteins. Preferably the protein may be selected from the following list: collagen, laminin, albumin, keratin, silk fibroin, fibronectin, or mixtures thereof.

In an embodiment, the composition may further comprise cell culture media or other buffered media.

In an embodiment, the composition may further comprise a hydrogel or a plurality of hydrogels. Preferably, wherein the hydrogel may be selected from a list consisting of carbopol, matrigel, hyaluronic acid, dextran, alginate, collagen, gellan gum, or mixtures thereof.

In an embodiment, the composition may further comprise an anti-inflammatory agent, an antiseptic agent, an antipyretic agent, an anaesthetic agent, a therapeutic agent, or mixtures thereof.

In an embodiment, the compositions may be combined with other excipients or active substances used in the context of veterinarian and human medicine.

In an embodiment, the composition may be administered by various routes. Including: topical, enteral and parenteral. Topical routes include application into the skin and mucous. Parenteral administration routes include intra-arterial, intra-articular, intracavitary, intracranial, epidural, intradermal, intralympathic, intramuscular, intraocular, intrasynovial, intravenous, or subcutaneous. Enteral routes include oral and gastro-intestinal.

In a preferred embodiment, dosage of the composition can be adapted to the administration route, as well as to the patient profile, including age, gender, condition, disease progression, or any other phenotypic or environmental parameters.

In a preferred embodiment, the composition may be in a solid form such as an amorphous, crystalline or semi-crystalline powder, granules, flakes, tablets, pills, scaffolds, capsules and suppositories. Such a solid form can be converted into a liquid form by mixing the solid with a physiologically appropriate liquid such as solvents, solutions, suspensions and emulsions.

In a preferred embodiment, the present invention provides a method for treating a patient (human or veterinarian) with regenerative medicine or tissue engineering, the method comprising administering an effective amount of chitosan/composition described above to the patient (human or veterinarian).

In a preferred embodiment, the present invention provides chitosan or its derivatives to use in regenerative medicine or tissue engineering (human or veterinarian). Moreover, the present invention provides the use of chitosan or its derivatives in the manufacture of a medicament for regenerative medicine or tissue engineering (human or veterinarian).

In a preferred embodiment, the invention provides the composition described above to use in human or veterinarian therapy. Further, the present invention provides the use of the composition described above in the manufacture of a medicament to use in human or veterinarian regenerative medicine or tissue engineering.

In a preferred embodiment, the invention provides the composition described above to use in human or veterinarian drug delivery.

In a preferred embodiment, the invention provides the composition described above to use in human or veterinarian cell delivery.

In a preferred embodiment, the invention provides the composition described above to use in human or veterinarian diagnosis.

Throughout the description and claims the word “comprise” and variations of the word, are not intended to exclude other technical features, additives, components, or steps. Additional objectives, advantages and features of the solution will become apparent to those skilled in the art upon examination of the description or may be learned by practice of the solution.

### BRIEF DESCRIPTION OF THE DRAWINGS

The following figures provide preferred embodiments for illustrating the description and should not be seen as limiting the scope of the present disclosure.

FIG. 1: Representative example of the FTIR spectra obtained for the studied chitosans. The characteristic bands related to the chitosan chemical structure are indicated.

FIG. 2: Representative example of the ¹H NMR spectra obtained for the studied chitosans. This spectrum corresponds to the chitosans obtained after the first reaction cycle. The characteristic peaks related to the chitosan chemical structure are indicated.

FIG. 3: Representative example of a SEC-MALLS chromatogram obtained for the chitosans of the present disclosure.

FIG. 4: Representative example of the ¹H NMR spectra obtained for the chitosans of the present disclosure. This spectrum corresponds to the chitosans from the second deacetylation cycle.

FIG. 5: Example of a membrane prepared by solvent-casting of a chitosan solution (0.5% in 2% acetic acid). A) Dried formulation and B) water re-hydrated formulation. C) Shows the scanning electron characterization of the dry membrane, while D) shows its energy dispersive X-ray spectrometry characterization.

FIG. 6: Comparison between solvent-casted chitosan solutions (0.1% in 2% acetic acid) three days after the casting. A) High molecular weight extracted chitosan of the present disclosure and B) medium molecular weight commercial chitosan.

FIG. 7: Comparison between solvent-casted chitosan (0.1% in 2% acetic acid) formulations. A) Membrane obtained using the high molecular weight extracted chitosan of the present disclosure and B) formless mass obtained using the medium molecular weight commercial chitosan.

FIG. 8: Example of a chitosan (0.5% in 2% acetic acid)-fucoidan hydrogel formed by ionic interaction. The colour of the hydrogel increases as it does the concentration of fucoidan: A) 2.5%, B) 5% and C) 10%.

FIG. 9: Comparison between the outcomes of chitosan (0.1% in 2% acetic acid)-fucoidan ionic interaction. A) Macro-hydrogels obtained using the high molecular weight extracted chitosan of the present disclosure and B) opaque solution obtained using the medium molecular weight commercial chitosan.

FIG. 10: Examples of membranes with a yellow colouring. The transparent chitosan membranes acquired this colour upon albumin loading.

### DETAILED DESCRIPTION

The present disclosure refers to the physicochemical and structural characterization of high molecular weight β-chitosan isolated from marine industry residues, more specifically from squid pens. It also refers to the application of this polymer and/or its derivatives in biomedicine.

In the present disclosure, Cht was extracted following a simpler procedure than those described in the literature. This extraction was performed in a shorter period of time and in a more eco-friendly manner when compared with conventional procedures, as less energy was utilized. More specifically, the received squid pens were gently washed with distilled water, to eliminate gross impurities. To achieve the greatest possible degree of reproducibility, the dried squid pens were milled (Ultra Centrifugal Mill ZM200, Retsch, Haan, Germany) and the obtained powder sieved (Analytical Sieve Shaker AS200, Retsch, Haan, Germany). Subsequently 5g of Cht powder with a particle size between 63 and 125 μm were added to 200 mL of reaction medium (50% NaOH). The collection was left to react during 2 hrs at 75° C., under constant magnetic stirring. This process was performed under N₂ atmosphere, in order to avoid the oxidation of the polysaccharide. Finally, the reaction product was abundantly washed with water until neutrality was reached.

In an embodiment, the obtained chitosan was frozen at −80° C. and freeze-dried for 3 days.

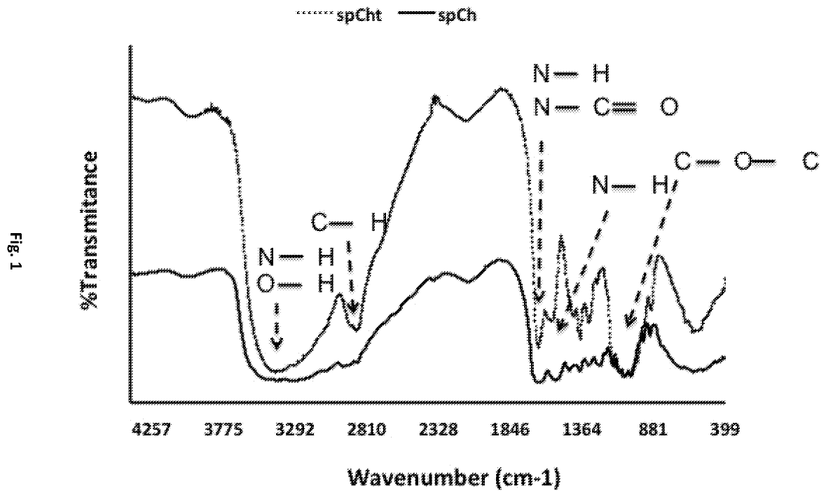

In an embodiment, the chemical structure of the obtained chitosan was characterized by FTIR spectroscopy. The FTIR spectrum was obtained using Shimadzu IRPrestige 21 spectromer (IRPrestige 21, Shimadzu, Europe). Samples were prepared as potassium bromide tablets at room temperature. The spectrum was collected by averaging 32 scans with a resolution of 4cm⁻¹, corresponding to the 4000-400 cm⁻¹ spectrum region. The obtained chitosan spectra displayed all the characteristic bands of chitosan. Indeed, all the studied samples lead to identical spectra. In this way, the spectrum of batch IV was selected as representative (FIG. 1). The peak at 3417 cm⁻¹ corresponds to O—H and N—H stretching vibrations, which is in concordance with the higher intensity of this peaks in the spectrum of chitosan with respect to that of chitin. The peak at 2879, related to C—H stretching vibrations, is more intense in the spectrum of chitosan than in the chitin spectrum. This particular difference between the two spectra has been previously described in the literature. The peaks ranging from 1560 to 1690 cm⁻¹ are attributed to the N—H bending vibration, while the band at 1695 cm⁻¹ is related to the N—H absorption in —NH₂. Again, this is in concordance with the higher intensity of this peaks in the spectrum of chitosan with respect to that of chitin. This strong absorption peak at 1695 cm⁻¹ may be also assigned to the C═O stretching vibration in the amide, due to partially acetylated amino groups. Finally, the peak at 1074 corresponds to the C—O—C stretching vibrations.

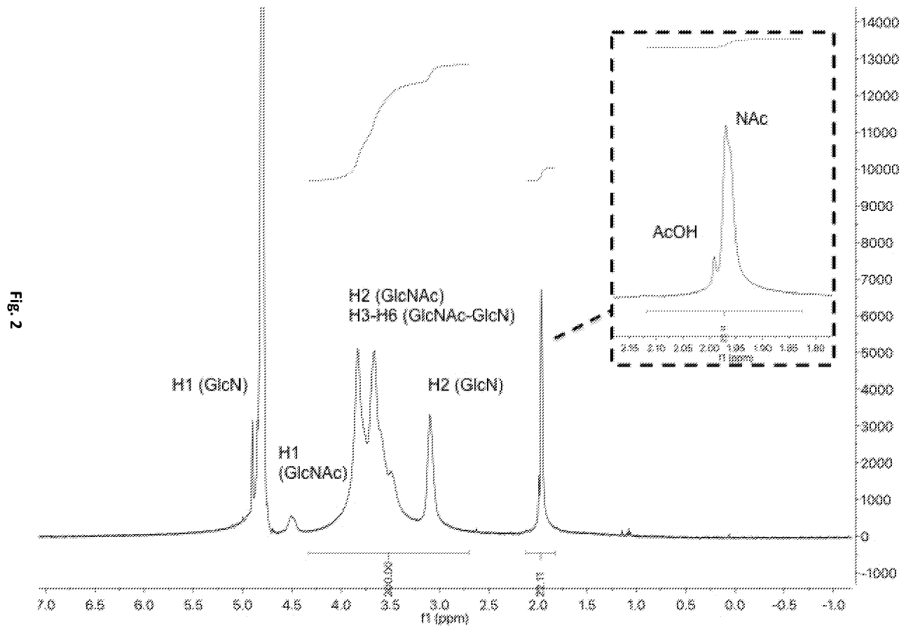

In an embodiment, the acetylation degree was determined by nuclear magnetic resonance (NMR). The ¹H-NMR spectra of chitosan was obtained in a 2% DCI solution in D₂O at 25° C., being recorded under the Burker Avance III spectral (Avance III HD 300 NMR-spectrometer, Bruker, Germany) conditions: resonance frequency of 400,13 MHz, with 1s pulse and 3.98 ms acquisition time. MestReNova Software 9.0 (Mestre-lab Research) was used for spectral processing. Chemical shifts are reported in ppm (δ). The NMR spectra confirmed that the product of the reaction was chitosan. The NMR spectra for all the studied samples were very similar. In this way, the spectrum of batch I was selected as representative (FIG. 2). The signal at 4.8 ppm coresponds to H1 GIcN, while the signal at 4.5 ppm corresponds to the H1 proton in GIcNA. The group of signals between 2.75 and 4.25 ppm correspond to the H2-H6 protons in the chitosan sugar. Meanwhile, the peak at 2 ppm corresponds to the 3 protons of the N-acetyl plus AcOH signals. The DA was determined from the relative integrals of this signal at 2 ppm, with respect to the combined H2-H6 protons. The NMR spectra also confirmed the good purification of the final product, as no signals from additional compounds were present in the different spectra.

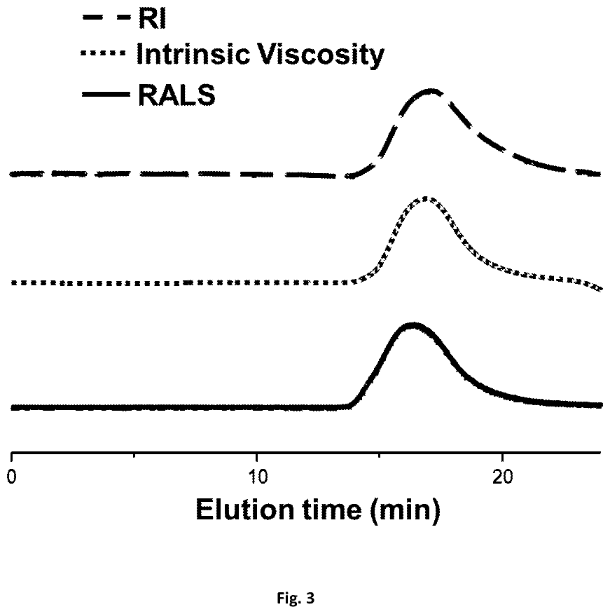

In an embodiment, chitosan was analysed by size exclusion chromatography-multiangle laser-light scattering. The SEC-MALLS method allows the determination of molecular weight and polydispersity. SEC-MALLS measurements were performed with a Viscotek TDA 305 (Malvern, United Kingdom) with refractometer, right angle light scattering and viscometer detectors on a set of four columns: pre-column Suprema 5 μm 8×50 S/N 3111265, Suprema 30 Å 5 μm 8×300 S/N 3112751, Suprema 1000 Å 5 μm 8×300 S/N 3112851 PL and Aquagel-OH MIXED 8 μm 7.5×300 S/N 8M-AOHMIX-46-51, with refractive index detection (RI-Detector 8110, Bischoff). A 0.15M NH₄OAc/0.2M AcOH buffer (pH=4.5) was used as eluent, at a rate of 1 mL/min. The chitosan samples were dissolved in this same buffer. The elution times and the RI detector signal were calibrated with a commercial calibration polysaccharide from Varian, Pullulan with Mp 47.1 kDa (Mw 48.8 kDa; Mn 45.5 kDa) and narrow polydispersity (1.07). Values of dn/dC were taken from the literature. Table 1 shows the values of different obtained parameters, among them, the Mw was 1,003.94±81.20 kDa. This is a higher molecular weight than that obtained following conventional procedures. The obtained SEC-MALLS chromatograms showed a single and symmetric peak, suggesting that there is a homogenous polysaccharide population (see a representative example in FIG. 3).

In an embodiment, the DA was further reduced, by submitting the product from the first reaction cycle to a new reaction cycle. For this purpose, all the content resultant from the previously described reaction cycle was mixed with 200 mL of reaction medium (50% NaOH). The system was left to react during 2 hrs at 75° C., under constant magnetic stirring. This process was performed under N₂ atmosphere. The reaction product was abundantly washed with water until neutrality was reached. The new DA was 5.66%±0.15 (see the spectrum in FIG. 4) and the reaction efficiency 80.34%±1.61.

TABLE 1
Values of RI area, Peak RV, Mn, Mw and Mw/Mn for the
studied chitosans, obtained after SEC-MALLS characterization.
A 0.15M NH4OAc/0.2M AcOH buffer (pH = 4.5) was
used both as dissolution buffer and as eluent.

| | RI area | Peak RV | Mn | Mw | |
|---|---|---|---|---|---|
| Sample* | (mvmL) | (mL) | (KDa) | (KDa) | Mw/Mn |
| Batch I | 285.672 ± | 16.609 ± | 567,234 ± | 1,066,341 ± | 1.655 ± |
| | 46.097 | 0.319 | 47,602 | 391,000 | 0.407 |
| Batch II | 348.972 ± | 17.971 ± | 573,434 ± | 1,040,581 ± | 1.818 ± |
| | 40.835 | 1.567 | 72,615 | 141,855 | 0.116 |
| Batch III | 354.408 ± | 18.093 ± | 573,056 ± | 1,023,845 ± | 1.800 ± |
| | 39.654 | 1.520 | 58,818 | 65,760 | 0.169 |
| Batch IV | 371.726 ± | 17.939 ± | 589,945 ± | 884,992 ± | 1.500 ± |
| | 46.981 | 1.011 | 45,279 | 76,501 | 0.051 |

*Sample concentration: 2 mg/mL

In an embodiment, the previous reaction was afterwards scaled. Accordingly, it was performed parting from 15 g of squid pens powder. The amounts of reagents were proportionally adjusted. The second reaction cycle was also performed. All the obtained results were very similar to those obtained before the scaling.

In an embodiment, membranes for tissue engineering or drug delivery applications were prepared by using the obtained chitosan. The membranes were prepared by solvent-casting. More specifically, 0.5% and 1% chitosan where dissolved in 2% acetic acid and casted over plastic Petri dishes. The solvent was left to evaporate at room temperature in an appropriate chamber. The resulting membranes were neutralized (0.1 M sodium hydroxide during 10 minutes). Water was utilized to eliminate sodium hydroxide residues.

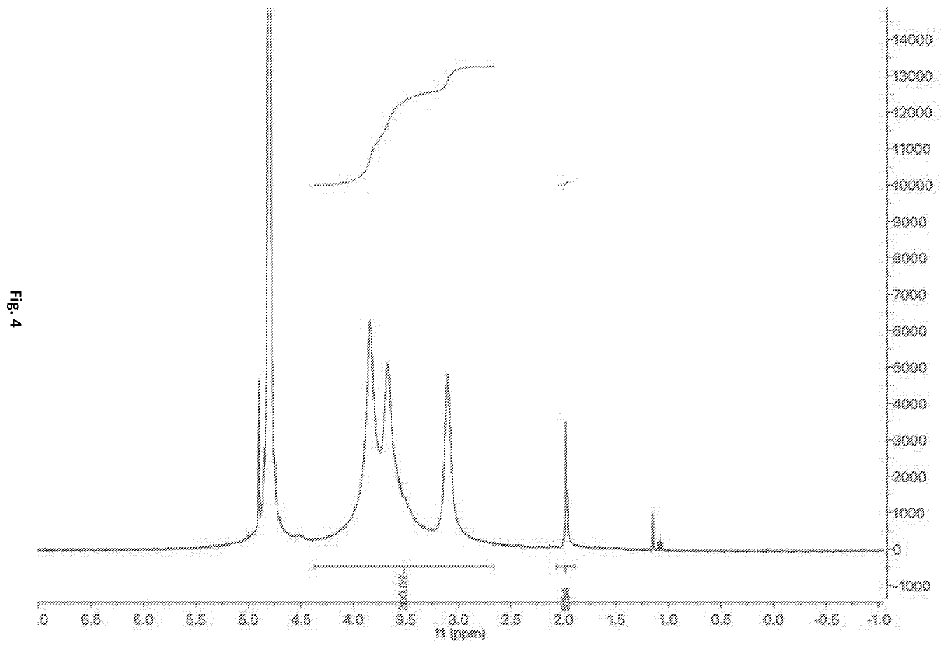

In an embodiment, with long-term storage in mind, the prepared membranes were oven-dryed (see the example in FIG. 5A). The water contact angle was 80.43°, which is in concordance with the literature. These membranes were characterized by scanning electron microscopy, showing a flat surface (see the example in FIG. 5C). Energy dispersive X-ray spectrometry characterization confirmed the presence of C, O and N in the membranes (see the example in FIG. 5D).

In an embodiment, thinking in application/administration after long-term storage, the prepared membranes were re-constituted by re-hydration with distilled water (see the example in FIG. 5B).

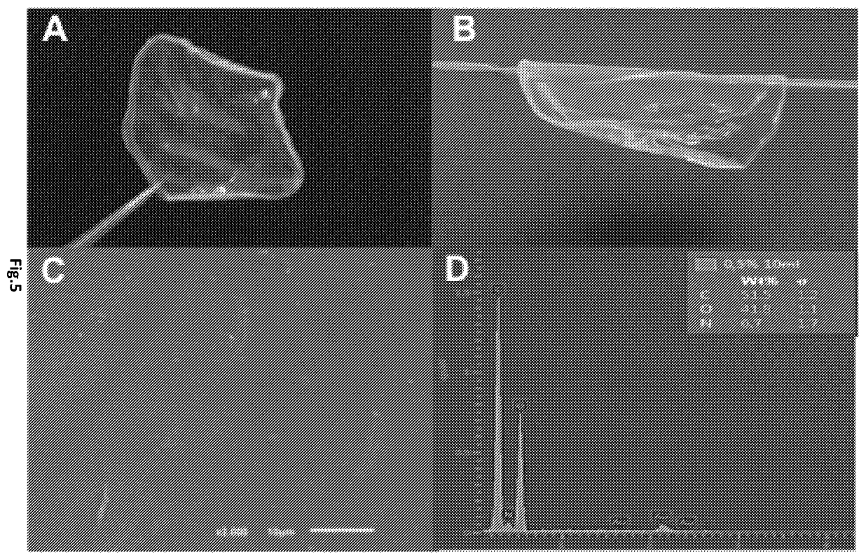

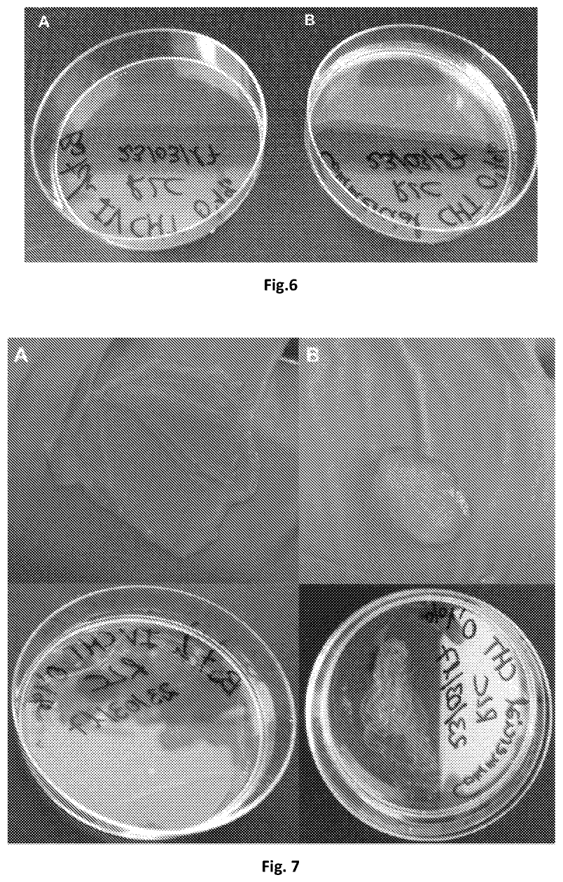

In an embodiment, membranes were obtained using lower chitosan concentrations (0.1%). The resultant formulations were compared to that obtained with medium molecular weight commercial chitosan, with a similar AD to that of the chitosan from the present invention. The procedure was the same as in the previous paragraph. Clear differences were observed between formulations. In the case of the present invention chitosan the membranes were formed faster (three days faster) (see FIG. 6). The authors relate this faster solvent evaporation to the high molecular weight of their chitosan, which provokes a specific arrangement of the chitosan chains, thus diminishing the entrapment of the water molecules. In addition, the formulation obtained with the present invention chitosan maintains indeed the form of a membrane (see the example in FIG. 7A), while the commercial chitosan gives rise to a formless mass (see the example in FIG. 7B).

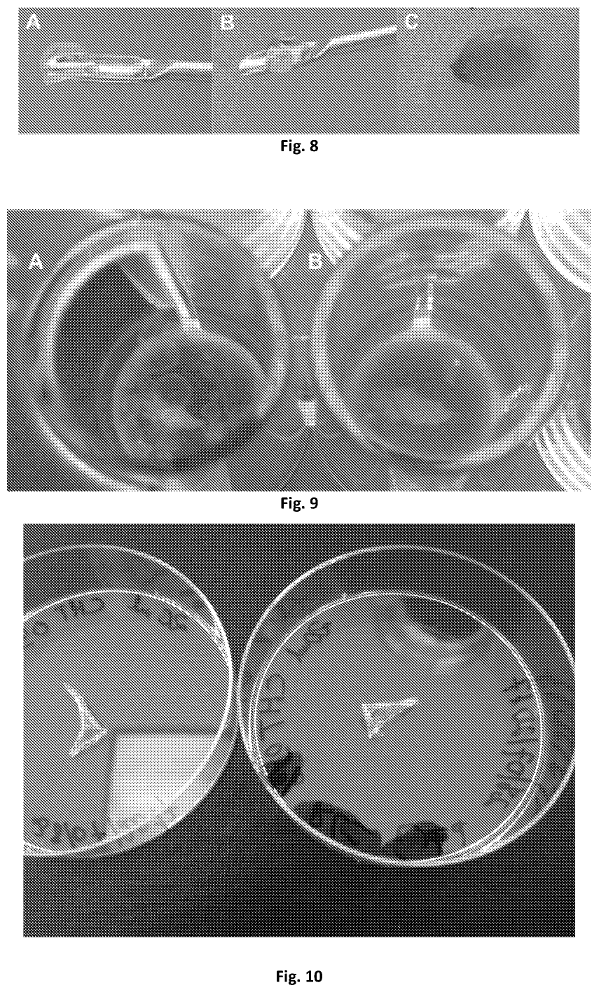

In an embodiment, hydrogels with potential for tissue engineering and drug delivery were prepared by using the obtained chitosan. These hydrogels were formed by electrostatic interaction with other polysaccharides i.e. chondroitin sulphate, fucoidan, gellan gum or alginate. More specifically, chitosan (concentrations 0.5 and 1% in 2% acetic acid) was mixed by mechanical agitation with the previously mentioned polymers (concentrations 2.5, 5 and 10% in water) at different rations. The gelation of the different formulations occurred immediately, and the hydrogels were neutralized (0.1 M sodium hydroxide during 10 minutes). Water was utilized to eliminate sodium hydroxide residues (see the example in FIG. 8).

In an embodiment, membranes were obtained using lower chitosan concentrations (0.1%). The resultant formulations were compared to that obtained with medium molecular weight commercial chitosan, with a similar AD to that of the chitosan from the present invention. The procedure was the same as in the previous paragraph. Clear differences were observed between formulations. The interaction between the present invention chitosan and the tested polysaccharide gave indeed rise to macro-hydrogels (see the example in FIG. 9A), while in the case of the commercial chitosan only solutions were observed (see the example in FIG. 9B).

In an embodiment, the amount of protein present in the extracted chitosan obtainable by the extraction method describe in the present disclosure was compared with that present in a previously purified commercial chitosan.

To assess the deproteinization efficiency of our methodology 10 mg of chitosan (with MW=1,003±81 kDa and a DA=22.8% (±0.7)) were immersed in 3 mL of milliQ water. The system was left under mechanical stirring for 48 hours. After this time a Micro BCA™ Protein Assay Kit (23235, ThermoFisher) was used for protein determination, following the instructions given by the supplier.

The same process was repeated with commercial chitosan (MW=190-310 kDa and an DA=18%, information provided by the supplier), after its purification in our lab.

These studies were performed in triplicate (n=3) and the results indicated as average±SD.

TABLE II
Protein content determined by microBCA analysis.

| Chitosan | Proteins (mg/mL) |
|---|---|
| Batch I | 0.029 ± 0.011 |
| Batch II | 0.052 ± 0.002 |
| Batch III | 0.075 ± 0.004 |
| Batch IV | 0.058 ± 0.023 |
| Commercial | 0.101 ± 0.02 |
| (MW, DA) | |

The results, presented in Table II, indicate a lower protein content in the extracted chitosan obtainable by the extraction method described in the present disclosure than in the commercial chitosan.

In an embodiment, it was proved the ability of the obtained chitosan to form membranes at low concentration. This property allows the use of the membranes of the present disclosure in medicine/biomedicine, namely tissue engineering. This interest grows if these membranes are able to act as drug delivery vehicles of bioactive molecules. Albumin was used as a model molecule to evaluate this ability.

Aliquots (50 μl) of 1, 2.5 and 5% albumin solution (PBS pH=7.4) were placed over 1 cm² membrane samples. The solution was left to penetrate the membrane for 1 hour at 37° C., point at which total solvent evaporation was observed. The supports where the membranes were placed during the loading step were washed with PBS (pH=7.4). These washing solutions were analyzed using a Standard UV-VIS Photospectrometry (UV-1601, Shimadzu, Australia), and the amount of albumin loaded within the membrane calculated utilizing Eq. 1:

% EE=[(iD−fD)/iD]*100, Eq. 1:

where iD is the incorporated drug and fD is the free drug.

In an embodiment, the loaded membranes were immersed in 3.5 mL of PBS solution (pH=7.4) and placed at 37° C. under mechanical stirring. After 0.5, 1 and 5 hours the release mediums were analysed using a Standard UV-VIS Spectrophotometry (UV-1601, Shimadzu, Australia). The release studies were not accumulative. The concentration of released drug was calculated with the help of an albumin calibration curve.

These studies were performed in triplicate (n=3) and the results indicated as average±SD.

In an embodiment, all the drug included within the loading solutions was effectively incorporated by the membranes. Indeed, after the loading the membranes (originally transparent) acquired the same colour as the albumin powder (yellow). This effect can be appreciated in FIG. 10.

The release of this protein was also very efficient. Indeed, all the albumin was released after half an hour of study. This is probably due to the high hydrophilicity of albumin. In addition, the experiment was really reproducible, with all the UV spectra overlapping. This indicates a very homogeneous and reproducible arrangement of the chitosan chains during the formation of the membranes.

Those skilled in the art will recognize or be able to ascertain using no more than routine experimentation, many equivalents to the specific embodiments of the invention described herein. The scope of the present invention is not intended to be limited to the above description, but rather is as set forth in the appended claims.

Where singular forms of elements or features are used in the specification of the claims, the plural form is also included, and vice versa, if not specifically excluded. For example, the term “a polysaccharide” or “the polysaccharide” also includes the plural forms “polysaccharides” or “the polysaccharides,” and vice versa. In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim.

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims.

The above-described embodiments are combinable.

The following claims further set out particular embodiments of the disclosure.

1. A method for obtaining a high molecular weight chitosan, comprising:
providing milled squid pen with a particle size between 63 and 125 μm; and
reacting NaOH with the milled squid pen particles selected in the previous step, for at least 1.5 hrs at 75° C., under stirring and in a N₂ atmosphere.

2. The method of claim 1, wherein the step of providing milled squid pen comprises milling squid pen and selecting the milled squid pen with a particle size between 63 and 125 μm.

3. The method of claim 2, wherein the amount of the selected milled squid pen particles is between 4-20 g

4. The method of claim 1, wherein the amount of NaOH is 200 mL.

5. The method of claim 1, wherein the NaOH is a solution of 50% (v/v) NaOH.

6. The method of claim 1, wherein the reaction time is between 1.5 and 3.5 hrs

7. The method of claim 1, further comprising:
freezing the obtained chitosan at −80° C. and/or freeze-drying the obtained chitosan for 3 days.

8. The method of claim 1, further comprising previously washing the squid pen to eliminate impurities.

9. A chitosan comprising a molecular weight of 500-1200 kDa and an acetylation degree between 5-40%.

10. (canceled)

11. The chitosan of claim 9, wherein the acetylation degree is between 5-25%

12. The chitosan of claim 9, wherein the acetylation degree is between 5-15%

13. The chitosan of claim 9, wherein a protein concentration of the chitosan is up to 0.1 mg/ml.

14. (canceled)

15. The chitosan of claim 13, wherein the molecular weight is 1000 kDa-1200 kDA.

16. (canceled)

17. The chitosan of claim 9, wherein the chitosan is β-chitosan.

18. (canceled)

19. (canceled)

20. (canceled)

21. (canceled)

22. (canceled)

23. A pharmaceutical composition comprising the chitosan of claim 9, and an active ingredient.

24. (canceled)

25. The composition of claim 23, wherein the composition comprises 0.1 to 50% of said chitosan.

26. The composition of claim 23, further comprising one or more additional polysaccharides, wherein the one or more additional saccharides are seaweed polysaccharides beta glucan, galactomannan, mucilage, cellulose, inulin, pullulan, dextrin, starch, glycosaminoglycans, or mixtures thereof.

27. The composition of claim 23, further comprising a protein, a growth factor, a digestive enzyme, a metabolic enzyme hormone, a drug, or mixtures thereof.

28. The composition of claim 27, wherein the protein is selected from the group consisting of: collagen, laminin, albumin, keratin, silk fibroin, fibronectin, and mixtures thereof.

29. The composition of claim 23, further comprising a cell culture media or a buffered media, wherein the cell culture media is a liquid, semi-solid, solid or gas cell culture media or a natural, synthetic or semi-synthetic cell culture media.

30. (canceled)

31. (canceled)

32. (canceled)

33. (canceled)

34. (canceled)
